(19) United States
(12) Patent Application Publication
(10) Pub. No.: US 2010/0221328 A1
(43) Pub. Date: Sep. 2, 2010

(54) SUSTAINED-RELEASE FORMULATIONS

(76) Inventors: Christian F. Wertz, Maple Grove, MN (US); Victoria Ann O'Neill, Maple Grove, MN (US); Stephen M. Berge, Maple Grove, MN (US)

Correspondence Address: VIKSNINS HARRIS & PADYS PLLP, P.O. BOX 111098, ST. PAUL, MN, 55111-1098, US

(21) Appl. No.: 12/650,149

(22) Filed: Dec. 30, 2009

Related U.S. Application Data
(60) Provisional application No. 61/142,117, filed on Dec. 31, 2008.

(51) Int. Cl. A61K 9/62; A61K 9/14; A61K 9/22; A61P 31/12; A61P 25/16; A61K 31/13

(52) U.S. Cl. 424/461; 424/495; 424/468; 514/662; 514/659

(57) ABSTRACT

Described herein are extended-release or sustained-release formulations suitable for highly soluble pharmacologically active compounds, for example, amantadine. Dosage units for providing extended release are provided by the present invention. In some embodiments, the dosage unit comprises a plurality of coated beads having drug layer and a coating layer, wherein the coating layer comprises an ethylcellulose polymer. In some embodiments, near zero-order release of the active compound is achieved.

### CROSS-REFERENCE TO RELATED APPLICATION(S)

This patent application claims the benefit of priority of U.S. application Ser. No. 61/142,117, filed Dec. 31, 2008, which application is herein incorporated by reference.

### BACKGROUND

Many compounds are administered to patients multiple times daily due to a lack of sustained-release formulations for those compounds. Such administration is not ideal due to, e.g., problems with patient compliance and control of symptoms. Thus, sustained-release formulations of compounds, such as amantadine hydrochloride, are needed.

### SUMMARY OF CERTAIN EMBODIMENTS OF THE INVENTION

Achieving sustained release of amantadine using conventional approaches has proven to be challenging at least in part due to the extremely high water solubility of the commonly dosed active compound, amantadine hydrochloride. However, as described herein, sustained-release (SR) formulations of amantadine hydrochloride have been successfully prepared. It is anticipated that compounds with high water solubility may also be similarly formulated to achieve SR formulations of those compounds.

Accordingly, certain embodiments of the present invention provide SR formulations. A SR formulation of amantadine offers improved patient compliance and control of symptoms due to less frequent dosing and may provide an improved side-effect profile.

Certain embodiments of the present invention provide a dosage unit for providing extended release of a highly soluble pharmacologically active compound, the dosage unit comprising a plurality of coated beads, wherein each coated bead comprises:

a bead core having a diameter of about 500 microns or larger,

a drug layer applied to the bead core, the drug layer comprising the highly soluble pharmacologically active compound; and

a coating layer substantially encapsulating the bead core and drug layer and adapted to retard release of the pharmacologically active compound when the coated beads are exposed to an aqueous or acidic environment, the coating layer comprising an ethylcellulose polymer.

In certain embodiments, the pharmacologically active compound is a salt of amantadine. In certain embodiments, the pharmacologically active compound is amantadine hydrochloride.

In certain embodiments, the dosage unit comprises about 50 mg to about 400 mg of amantadine hydrochloride, and more suitably from about 100 mg to about 200 mg of amantadine hydrochloride.

In certain embodiments, the pharmacologically active compound is a salt of rimantadine.

In certain embodiments, from about 10 wt.-% to about 50 wt.-% (e.g., about 20, 30, or 40 wt.-%) of the coated bead is the pharmacologically active compound.

In certain embodiments, the bead core is an inert sphere. In certain embodiments, the bead core is a microcrystalline cellulose seed core. In certain embodiments, the bead core is a sugar sphere.

In certain embodiments, the dosage unit comprises bead cores having a diameter from about 710 to about 850 microns.

In certain embodiments, from about 50 wt.-% to about 95 wt.-% (e.g., about 60, 70, 75, 80, 85, 90, 95 wt.-%; e.g., at least about 90 wt.-%) of the drug layer is the pharmacologically active compound.

In certain embodiments, the drug layer comprises a binder. In certain embodiments, the binder comprises polyvinylpyrrolidone, hydroxypropyl methylcellulose, hydroxypropyl cellulose, or a combination thereof. In certain embodiments, the binder is polyvinylpyrrolidone.

In certain embodiments, the drug layer consists essentially of the pharmacologically active agent and binder.

In certain embodiments, from about 10 wt.-% to about 30 wt.-% of the coated bead is the coating layer.

In certain embodiments, the coating layer comprises a plasticizer. In certain embodiments, the plasticizer comprises glycerine, acetyltriethyl citrate, triethyl citrate, acetyltributyl citrate; dibutylsebacate, triacetin, triacetin citrate, polyethylene glycol, propylene glycol, or diethyl phthalate. In certain embodiments, the plasticizer is diethyl phthalate.

In certain embodiments, the coating layer consists essentially of the ethylcellulose polymer and plasticizer.

In certain embodiments, the coating layer retards release of the pharmacologically active compound when the coated beads are exposed to an aqueous or acidic environment such that not more than 50 wt.-% of the total pharmacologically active compound of the dosage unit is released into the environment within 4 hours.

In certain embodiments, at least 80 wt.-% of the total pharmacologically active compound of the dosage unit is released into the environment within 24 hours. In certain embodiments, at least 80 wt.-% of the total pharmacologically active compound of the dosage unit is released into the environment within 12 hours. In certain embodiments, at least 80 wt.-% of the total pharmacologically active compound of the dosage unit is released into the environment within 8 hours.

In certain embodiments, the dosage unit is in the form of a capsule, a tablet, or a sachet.

Certain embodiments of the present invention provide a method for providing a dosage unit that provides extended release of a highly soluble pharmacologically active compound, the dosage unit comprising a plurality of coated beads, the method comprising the steps of:

applying a drug layer to a plurality of bead cores, each having a diameter of about 500 microns or larger, wherein the drug layer comprises the pharmacologically active compound; and

encapsulating the drug layer with a coating layer to provide the coated beads, the coating layer adapted to retard release of the pharmacologically active compound when the coated beads are exposed to an aqueous or acidic environment, wherein the coating layer comprises an ethylcellulose polymer.

In certain embodiments, the method further comprises the steps of:

combining the plurality of coated beads with excipients suitable for tabletting to form a mixture; and

compressing the mixture into a tablet.

In certain embodiments, the method further comprises the steps of dispensing the plurality of coated beads into a gelatin capsule.

In certain embodiments, the method further comprises the steps of dispensing the plurality of coated beads into a sachet.

In certain embodiments, the drug layer is applied by:

spraying a solution onto the bead cores, the solution comprising the pharmacologically active compound, a binder, and a suitable solvent, and

evaporating the solvent to provide the drug layer.

In certain embodiments, the solvent is evaporated during the spraying step. In certain embodiments, the solvent is evaporated subsequent to the spraying step.

In certain embodiments, the coating layer is applied by:

spraying a solution onto the bead cores, the solution comprising the ethylcellulose polymer, a plasticizer, and a suitable organic solvent system, and

evaporating the organic solvent system to provide the coating layer.

In certain embodiments, the organic solvent system is evaporated during the spraying step. In certain embodiments, the organic solvent system is evaporated subsequent to the spraying step.

Certain embodiments of the present invention provide a method of treating a patient with Parkinson's Disease, comprising administering to the patient a dosage unit as described herein.

Certain embodiments of the present invention provide a method of treating a patient with a viral infection, comprising administering to the patient a dosage unit as described herein.

Certain embodiments of the present invention provide a method of treating a patient suffering from fatigue associated with multiple sclerosis, comprising administering to the patient a dosage unit as described herein.

In certain embodiments, the method comprises administering to the patient the dosage unit at most twice per day.

In certain embodiments, the method comprises administering to the patient the dosage unit at most once per day.

In certain embodiments, the administration provides a zero-order dissolution profile of the biologically active compound that releases at a constant rate over 24 hours.

In certain embodiments, the administration provides a zero-order dissolution profile of the biologically active compound that releases at a constant rate over 12 hours.

In certain embodiments, the administration provides a zero-order dissolution profile of the biologically active compound that releases at a constant rate over 8 hours.

In certain embodiments, SR formulations of amantadine, e.g., amantadine HCl, demonstrate zero-order release kinetics, e.g., over about 24 hours.

### BRIEF DESCRIPTION OF THE FIGURES

FIG. 1 depicts dissolution testing of the coated beads. Amantadine dissolution (plotted as % Label claim versus time) in water for each of the sustained-release systems (at 20% weight gain for each polymer system) is shown in FIG. 1.

FIG. 2 depicts data of coated beads including an ethylcellulose polymer sprayed from an organic solution (95% absolute ethanol/5% water; total solids 12.5% by weight) that exhibit slower release than coated beads including ethylcellulose sprayed from an aqueous dispersion.

FIG. 3 depicts dissolution indicating that the data does trend with ethylcellulose molecular weight, indicating that longer polymer chains are able to form a coating more resistant to amantadine release.

FIG. 4 depicts dissolution results for each of the coated bead lots. The finer grades of coated beads showed much faster release of amantadine when compared to coarser grades. The data suggest that bead particle size distribution is likely an important factor in providing adequate release retardation for a highly soluble active such as amantadine hydrochloride.

FIG. 5 depicts additional release profiles for drug-loaded CELPHERE® MCC CP-708 bead cores coated with an ethylcellulose-based coating (ETHOCEL®10) to varying weight gains. As FIG. 5 demonstrates, the coating layer weight gain (which relates to coating thickness) has a significant impact on amantadine release, with increasing weight gains showing slower release.

FIG. 6 depicts dissolution profiles of identical ethylcellulose-based coating layers (ETHOCEL®10) on sugar spheres (Lot 224.060) and CELPHERE® MCC bead CP-507 (Lot 224.099). The coated bead lots differ in substrate type only.

FIG. 7 depicts dissolution results to six months for a first formulation.

FIG. 8 depicts dissolution results to six months for a second formulation.

FIG. 9 depicts dissolution results to six months for a third formulation.

### DETAILED DESCRIPTION

There are currently no SR amantadine products on the market. Immediate release (IR) amantadine is dosed every 12 hours. As such, a once-daily, SR formulation may provide improved patient compliance and/or efficacy. Thus, the formulations described herein provide physicians with a more convenient dosing regimen for amantadine. A SR dosage form of amantadine should result in plasma concentrations of amantadine that are more stable over time than what is observed for the immediate release (IR) product. Such formulations should provide more consistent blood levels of amantadine and a reduced side-effect profile.

Certain embodiments of the present invention provide a dosage unit for providing extended release of a highly soluble pharmacologically active compound (such as amantadine hydrochloride). The dosage unit comprises a plurality of coated beads, wherein each coated bead includes a bead core having a diameter of about 500 microns or larger, a drug layer applied to the bead core, and a coating layer substantially encapsulating the bead core and drug layer, the coating layer comprising an ethylcellulose polymer. The coating layer is adapted to retard release of the pharmacologically active compound when the coated beads are exposed to an aqueous or acidic environment, either in vitro or in vivo.

In certain embodiments, the formulations combine the features of a coating layer composition and bead core particle size to achieve the desired release profile.

In the practice of the present invention, it was surprising that an ethylcellulose-based coating (ETHOCEL®) would retard the release of amantadine hydrochloride and provide the desired sustained release to a greater extent than a methacrylate-based coating (EUDRAGIT®) or a PVA-PVP copolymer-based coating (KOLLICOAT®). It was unexpected that coatings of ethylcellulose would prove significantly more effective than methacrylate- and polyvinylalcohol-based coatings at retarding the release of amantadine HCl from drug-loaded beads.

It was also surprising that that using a larger bead core as a substrate would provide approximate zero-order release over a 24-hour period of amantadine from coated beads containing a high drug load and an ethylcellulose-based coating layer. It was surprising that increasing the bead core size would have a dramatic impact on the amantadine release profile.

Experimental data provided herein demonstrates that the bead core size, coating layer composition, and coating layer thickness, and combinations thereof, are significant parameters for providing a desired dissolution profile for amantadine. Use of smaller bead core sizes results in much faster release profiles that are not zero-order. Similarly, use of coating layers that are not ethylcellulose-based results in faster release profiles that are not zero-order. The ethylcellulose-based coating (ETHOCEL®) grade used in the coating layer, and coating layer thickness, are also important parameters for achieving a desired release profile.

### Bead Core

In the practice of certain embodiments of the present invention, the dosage unit comprises a plurality of coated beads, wherein each coated bead includes a bead core having a diameter of about 500 microns or larger.

Different types of substrates, e.g., inert substrates, can be used as bead cores. In certain embodiments, the bead cores are nonpareils or sugar spheres. In certain embodiments, the bead cores are spheronized microcrystalline cellulose (MCC). In the practice of the invention, similar release profiles have been demonstrated using different substrates of the same size (sugar spheres 30/35 and CELPHERE® CP-507 MCC beads) with an ethylcellulose-based coating.

### Drug Layer

In the practice of certain embodiments of the present invention, a drug layer is applied to the bead core. The drug layer comprises a highly soluble pharmacologically active compound.

Suitable active compounds include compounds that are highly soluble, i.e., compounds that are “freely soluble” or “very soluble” in water, as described in the table below. In certain embodiments, the formulations may include compounds that are “freely soluble”. In certain embodiments, the formulations may include compounds that are “very soluble”.

| | Very | Freely | | Sparingly | Slightly | Very Slightly | Practically |
|---|---|---|---|---|---|---|---|
| | Soluble | Soluble | Soluble | Soluble | Soluble | Soluble | Insoluble |
| Solubility (S) | 1000 > S | 1000 > S > 100 | 100 > S > 33 | 33 > S > 10 | 10 > S > 1 | 1 > S > 0.1 | S < 0.1 |
| range (mg/mL) | | | | | | | |

In certain embodiments, the active compound is a compound that is a small molecule, is crystalline, has a MW<400 and is the salt of a weak base.

In certain embodiments, the active compound is amantadine or a salt thereof, e.g., amantadine HCl.

Amantadine hydrochloride is designated generically as amantadine hydrochloride and chemically as 1-adamantanamine hydrochloride. Amantadine hydrochloride is a stable white or nearly white crystalline powder, freely soluble in water and soluble in alcohol and in chloroform. Amantadine hydrochloride has pharmacological actions as both an anti-Parkinson and an antiviral drug.

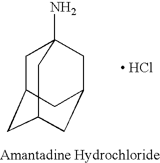

One suitable method for applying a drug layer to bead cores is by spray coating, such as fluidized bed coating. A fluidized bed technique generally employs a solution in which the active compound is dissolved. The solution may comprise a binder and/or other adjuvants as well. The solution is sprayed onto bead cores, and then the solvent is evaporated to leave the drug layer on the bead cores.

Conventional techniques other than fluidized bed coating (e.g., extrusion spheronization, spray drying, spray chilling, melt granulation) can also be used to apply the drug layer to bead cores.

### Binders

In the practice of certain embodiments of the present invention, a binder can be used in the drug layering process, generally by including the binder with the active compound in a coating solution. Such binders are well known in the art, and include such substances as hydroxypropylcellulose (HPC), hydroxypropyl methylcellulose (HPMC), microcrystalline cellulose, polyvinylpyrrolidone (PVP), polyethylene oxide, starch, methylcellulose, carboxymethyl cellulose, sucrose solution, dextrose solution, acacia, tragacanth and locust bean gum, and combinations thereof.

### Coating Layer

In the practice of certain embodiments of the invention, the coating layer substantially encapsulates the bead core and drug layer, and comprises an ethylcellulose polymer. The coating layer is adapted to retard release of the pharmacologically active compound when the coated beads are exposed to an aqueous or acidic environment, either in vitro or in vivo.

An example of a suitable ethylcellulose polymer is ETHOCEL®. All ETHOCEL® grades used herein were ETHOCEL® PREMIUM, which is supplied as a powder.

Methods for using ethylcellulose-based coatings are known in the art (see, e.g., “ETHOCEL® Ethylcellulose Polymers Technical Handbook”, The Dow Chemical Company, Form No. 192-00818-0905×AMS, September 2005, e.g., at pages 20-21).

It was surprisingly discovered that an ethylcellulose-based coating layer (e.g., ETHOCEL®) retards the release of amantadine and provides the desired sustained release to a greater extent than a methacrylate-based coating (EUDRAGIT®) or a PVA-PVP copolymer-based coating (KOLLICOAT®). In certain embodiments, the ethylcellulose-based coating layer is ETHOCEL®.

One suitable method for applying a coating layer is by spray coating, such as fluidized bed coating. A fluidized bed technique generally employs a solution in which the components (such as an ethylcellulose polymer) of the coating layer is dissolved. The solution may comprise a plasticizer and/or other adjuvants as well. The solution is sprayed onto drug-loaded bead cores, and then the solvent is evaporated provide a coating layer that substantially encapsulates the bead core and drug layer.

Solvents are generally used in the application of a coating layer. Ethylcellulose-based coating layers are generally best applied using an organic solvent system. One suitable solvent system employed herein is a solution of 95% EtOH/5% water. (As used herein, an “organic solvent system” encompasses solvent systems that have no more than a minor portion of water. A higher proportion of water would likely render the ethylcellulose polymer insoluble.) Other solvents that can be used include ethanol, methanol, acetone, chloroform and ethanol blend, ethyl lactate and ethanol blend, methyl salicylate and ethanol blend, methyl salicylate, toluene and ethanol blend, and methylene chloride and ethanol blend.

Conventional techniques other than fluidized bed coating (e.g., spinning disk coating, solvent evaporation, melt processing) can also be used to apply the coating layer.

Other solvents that can be used include ethanol, methanol, acetone, chloroform and ethanol blend, ethyl lactate and ethanol blend, methyl salicylate and ethanol blend, methyl salicylate, toluene and ethanol blend, and methylene chloride and ethanol blend. A solution of the ethylcellulose-based coating ETHOCEL® may be made with a solvent and a plasticizer. An example of an ethylcellulose-based coating is ETHOCEL®. Methods for using ethylcellulose-based coatings are known in the art (see, e.g., “ETHOCEL® Ethylcellulose Polymers Technical Handbook”, The Dow Chemical Company, Form No. 192-00818-0905×AMS, September 2005, e.g., at pages 20-21).

### Plasticizers

In the application of a coating layer, a coating solution may comprise a plasticizer along with the ethylcellulose polymer. Plasticizer type and level can be varied, as appropriate, to optimize product performance. Suitable plasticizers include glycerine, acetyltriethyl citrate, triethyl citrate, acetyltributyl citrate; dibutylsebacate, triacetin, triacetin citrate, polyethylene glycol, propylene glycol, diethyl phthalate and the others known in the art.

### Formulations

Coated beads according to certain embodiments of the invention may be filled into sachets or capsules, or compressed into tablets, for example.

Formulations according to certain embodiments of the present invention may be systemically administered, e.g., orally, in combination with a pharmaceutically acceptable vehicle such as an inert diluent or carrier. They may be enclosed in gelatin capsules, may be compressed into tablets, or provided in a sachet. For oral therapeutic administration, the formulation may be combined with one or more excipients and used in the form of ingestible tablets, buccal tablets, troches, capsules, wafers, and the like. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate. Various other materials may be present as coatings or to otherwise modify the physical form of the solid unit dosage form. For instance, tablets, pills, or capsules may be coated with gelatin, wax, shellac or sugar and the like. Any material used in preparing any unit dosage form should be pharmaceutically acceptable and substantially non-toxic in the amounts employed.

### Analytical Procedures

Amantadine HCl is highly soluble in water or simulated gastric fluid and lacks a chromophore. Both of these properties make analytical measurements related to drug release challenging.

Prior to dissolution testing, all samples of coated beads were passed through a 16-mesh sieve to remove any large aggregates and retained on a 30-mesh sieve to remove any fines. Dissolution testing of coated beads was performed using USP Apparatus 1 (baskets) at 100 rpm. Dissolution media was water (900 ml) maintained at 37±0.5° C. Sample size varied and was dependent upon the specific composition of the coated beads to be tested. Generally a suitable mass of coated beads was used to correspond to a fixed dose of the analyte.

At each specified time point, approximately 10 mL of media was removed using a stainless steel cannula and plastic syringe. (Media replacement was not performed.) Media samples were filtered using porous (full flow) filters (QLA, Inc., Bridgewater, N.J., part number FIL010-01).

A memantine internal standard was prepared by dissolving a known quantity of memantine standard in methanol to achieve a final concentration of ≈0.4 mg/ml. Each media sample was diluted with an equal volume of memantine internal standard solution to result in a memantine internal standard concentration of ≈0.2 mg/ml.

A chromatographic standard was prepared with the final concentration of amantadine at ≈0.2 mg/mL and memantine at ≈0.2 mg/ml. Solvent composition was the same between standards and samples (50/50, % v/% v, methanol/water).

The analytical method used to evaluate dissolution media samples utilized a gas chromatograph (HP 5890 series II) with a flame ionization detector and Zebron ZB-5 capillary GC column. Column dimensions are 30 meters X 0.53 mm internal diameter with a 5 μm film thickness (Phenomenex part number 7HK-G002-39). The GC operated in direct injection mode using a Restek Cyclo-Uniliner Liner (4 mm×6.3×78.5 mm, Siltek Deactivated, part number 20338-214.5). The carrier gas was helium in a constant flow mode at 3.0 psi with the sample inlet held at 220° C. Injection volume was 1 μL. The FID detector was held at 250° C. Chromatographic run time was 20 minutes. After sample injection, oven temperature was initially 140° C. and was held for 15 minutes. At 15 minutes, a temperature ramp increased oven temperature at 20° C./minute over five minutes. Final oven temperature was 240° C. Every six samples were bracketed by chromatographic standards. Calculations were done using peak area ratios (amantadine area/memantine internal standard area). Drug released at each time point included corrections for changes in vessel volume and the amount of drug removed during previous sample pulls.

The United States Pharmacopeia chromatographic method for amantadine hydrochloride involves a basic extraction using sodium hydroxide and dichloromethane to remove the drug from an aqueous phase. The novel use of direct injection GC with the memantine internal standard avoids the tedious extraction procedure of the compendial method and allows for rapid and accurate analysis of dissolution samples. Baseline resolution between the amantadine and memantine ensures the integrity of the GC procedure.

The invention will now be illustrated by the following non-limiting Examples.

### Example 1

### Sustained-Release of Amantadine

In preparing drug-loaded beads, polyvinylpyrrolidone (PVP) was used as the binder due to its excellent binding properties and its wide use in similar applications. It was estimated experimentally that the aqueous solubility of amantadine hydrochloride was greater than 500 mg/mL (based on visual observations). A coating solution for drug layering was prepared having a drug:polymer ratio of 10:1 and a solution viscosity suitable for spraying:

| | |
|---|---|
| 32.0% w/w | Amantadine HCl |
| 3.2% w/w | PVP (KOLLIDON ® 30) |
| 64.8% w/w | Water |

Several batches of drug-loaded beads were prepared by fluidized bed coating, and subsequently combined (dry-blended together) to support several subsequent experiments. In all cases, sugar spheres (30/35 mesh) served as the inert substrate. The target drug load was 50% w/w and was confirmed for all lots via gas chromatography. (A “drug load” of 50% w/w in this context indicates that 50% by weight of the drug-loaded beads is the active compound.)

Coating trials were designed in order to investigate several common sustained-release polymer systems: an ethylcellulose-based coating (ETHOCEL®10), an aqueous ethylcellulose dispersion (AQUACOAT®ECD), a methacrylate-based coating (EUDRAGIT® RS 30D), and a PVA-PVP copolymer-based coating (KOLLICOAT® SR30D).

Drug-loaded beads were coated by fluidized bed coating, to a final weight gain of 20% w/w, with samples also withdrawn from the coating chamber at weight gains of 5, 10, and 15% w/w. (A “weight gain” of 20% w/w in this context indicates that the coated beads are 20% heavier by weight after coating as compared to the drug-loaded beads before coating.) Detailed information on each of these systems can be found in the composition table below (Note: all composition tables listed as % w/w).

| Lot # | 224.043 | 224.064 | 224.047 | 224.060 |
|---|---|---|---|---|
| Substrate | 83.33 | 83.33 | 83.33 | 83.33 |
| Amantadine HCl | 41.67 | 41.67 | 41.67 | 41.67 |
| PVP K30 | 4.17 | 4.17 | 4.17 | 4.17 |
| Sugar Sphere 30/35 | 37.50 | 37.50 | 37.50 | 37.50 |
| Coating | 16.67 | 16.67 | 16.67 | 16.67 |
| Ethocel 10 | | | | 13.34 |
| Aquacoat | | | 15.15 | |
| Eudragit RS 30D | 9.80 | | | |
| Kollicoat SR30D | | 11.49 | | |
| TEC | 1.96 | 1.15 | | |
| DEP | | | 1.52 | 3.33 |
| Talc | 4.90 | 4.02 | | |

The composition of each coating system was chosen in part based on each manufacturer's recommendation. As a result, not all coating systems have the same plasticizer type and/or level. In addition, the methacrylate-based coating (EUDRAGIT®) and the PVA-PVP copolymer-based coating (KOLLICOAT®) systems require significant talc levels in the formulation to prevent aggregation due to tackiness associated with these polymers. In all cases, no pore formers or diffusion-enhancing polymers were added to the coating systems.

A methacrylate-based coating (EUDRAGIT®), a PVA-PVP copolymer-based coating (KOLLICOAT®), and an ethylcellulose aqueous dispersion (AQUACOAT®) systems all require an additional curing step, post coating. Curing is necessary to allow the polymer chains time to relax on the bead surface, resulting in more uniform, rate-controlling coatings. The curing process for each system was conducted in a small oven using the following target conditions, per manufacturer's recommendation:

| | Temp | Time |
|---|---|---|
| Coating System | (° C.) | (hr) |
| Eudragit RS 30D | 50 | 24 |
| Kollicoat SR30D | 70 | 0.25 |
| Aquacoat ECD | 60 | 2 |

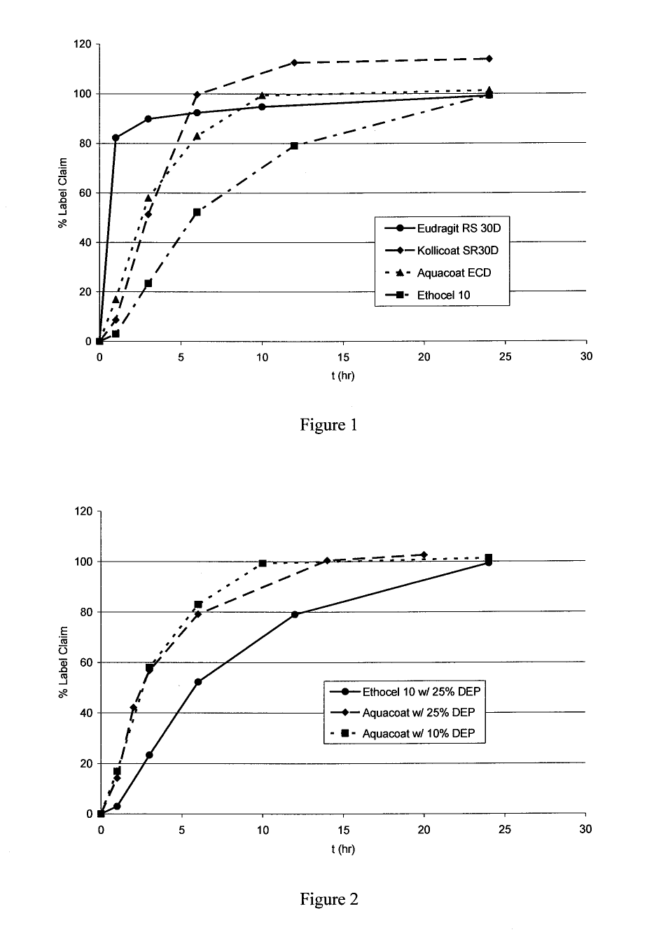

Dissolution testing of the resulting coated beads was performed according to the method described above. Amantadine dissolution (plotted as % Label claim versus time) in water for each of the sustained-release systems (at 20% weight gain for each polymer system) is shown in FIG. 1.

It is clear from FIG. 1 that none of these coated beads exhibit zero-order release of amantadine. However, there is a strong dependence of amantadine release rate on the type of polymer in the coating layer. A methacrylate-based coating (EUDRAGIT® RS 30D) provided the least resistance, with more than 80% amantadine released after only 1 hour. A PVA-PVP copolymer-based coating (KOLLICOAT® SR30D) and the ethylcellulose aqueous dispersion (AQUACOAT®ECD) provided somewhat better release retardation, releasing ˜80% amantadine between 5 and 6 hours. The ethylcellulose-based coating ETHOCEL® 10 provided the most effective release barrier, releasing 80% amantadine after 12 hours.

A methacrylate-based coating (EUDRAGIT®) controls the rate of drug release via a diffusion-controlled model. Conversely, an ethylcellulose-based coating (ETHOCEL®) controls the rate of drug release osmotically. The rapid release of amantadine from the coated beads employing the methacrylate-based coating layer suggests that a pure, diffusion-controlled polymer coating is not adequate to significantly retard the release rate for a compound with an aqueous solubility as large as amantadine hydrochloride.

Another consideration is the coating solution itself. The ethylcellulose-based coating ETHOCEL® was sprayed from an organic solution (95% absolute ethanol/5% water; total solids 12.5% by weight), which may explain its enhanced performance when compared to the other three systems, all of which were sprayed as aqueous colloidal dispersions of un-dissolved polymer. Each of the dispersions requires a secondary curing step to allow the un-dissolved polymer particles to coalesce into a uniform coating. Although each of the dispersion systems were cured prior to dissolution testing, this process was not investigated and/or optimized and may help to explain the rapid release of amantadine using these polymers.

Conversely, the ethylcellulose-based coating sprayed from an organic solution might more readily form a uniform coating during the coating process, with no curing step required.

To investigate this further, a series of dissolution profiles for the ethylcellulose-based coating ETHOCEL® and the ethylcellulose aqueous dispersion (AQUACOAT®) systems in which diethyl phthalate (DEP) has been used as the plasticizer (the compositions for each is found in the table below) were generated. Each of the ethylcellulose aqueous dispersion (AQUACOAT®) systems was cured following coating using the conditions listed above. (Note: the ethylcellulose aqueous dispersion AQUACOAT® was sprayed to a final weight gain of only 19.6% w/w due to an inadequate amount of coating solution for lot # 224.033.)

| | Lot # | | |
|---|---|---|---|
| | 224.060 | 224.033 | 224.047 |
| Substrate | 83.33 | 83.61 | 83.33 |
| Amantadine HCl | 41.67 | 41.81 | 41.67 |
| PVP K30 | 4.17 | 4.18 | 4.17 |
| Sugar Sphere 30/35 | 37.5 | 37.63 | 37.5 |
| Coating | 16.67 | 16.39 | 16.67 |
| Ethocel 10 | 13.34 | | |
| Aquacoat | | 13.04 | 15.15 |
| DEP | 3.33 | 3.35 | 1.52 |

As evidenced by FIG. 2, coated beads comprising the ethylcellulose polymer sprayed from an organic solution (95% absolute ethanol/5% water; total solids 12.5% by weight) exhibit slower release than coated beads comprising ethylcellulose sprayed from an aqueous dispersion. This result holds true for the aqueous dispersion AQUACOAT® at two different plasticizer levels.

### Example 2

### Molecular Weight of Ethylcellulose in Coating Layer

To determine the impact of the ethylcellulose-based coating (ETHOCEL®) molecular weight on amantadine release rate, a series of coated beads was manufactured using increasing ethylcellulose-based coating molecular weights (ETHOCEL® grades 10, 45, 100). Each sample of drug-loaded beads was coated to a target weight gain of 20% w/w, resulting in compositions differing only in polymer chain lengths for the ethylcellulose coating layer. Formulation compositions and dissolution results are shown below.

| | Lot # | | |
|---|---|---|---|
| | 224.037 | 224.055 | 224.051 |
| Substrate | 83.33 | 83.33 | 83.33 |
| Amantadine HCl | 41.67 | 41.67 | 41.67 |
| PVP K30 | 4.17 | 4.17 | 4.17 |
| Sugar Sphere 30/35 | 37.5 | 37.5 | 37.5 |
| Coating | 16.67 | 16.67 | 16.67 |
| Ethocel 10 | 13.89 | | |
| Ethocel 45 | | 13.89 | |
| Ethocel 100 | | | 13.89 |
| TEC | 2.78 | 2.78 | 2.78 |

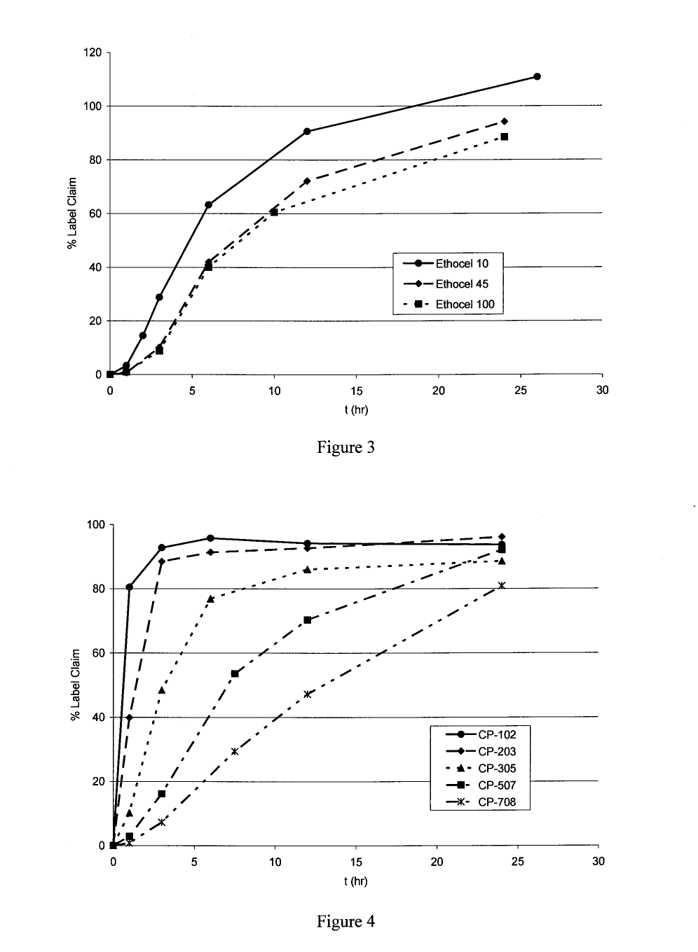

The dissolution data does trend with ethylcellulose molecular weight, indicating that longer polymer chains are able to form a coating more resistant to amantadine release (see FIG. 3). Increasing the ETHOCEL® grade to 45 and 100 resulted in the delay of 80% amantadine release to between 16 and 20 hours (compared to 10 hours for the ethylcellulose-based coating ETHOCEL®10).

The data suggest that there is a molecular weight threshold, beyond which no further retardation of amantadine release is achieved. It is also important to note that coating solutions using the ETHOCEL®45 and ETHOCEL®100 grades were prepared using very low solids content (resulting in excessive spray times) due to the extremely high solution viscosities associated with these polymer grades.

Furthermore, there was still significant curvature to the dissolution profiles, indicating that zero-order release had not been achieved.

### Example 3

### Bead Core Size

A series of coated beads was manufactured using spheronized microcrystalline cellulose (MCC) as the substrate. CELPHERE® MCC beads are supplied in five different grades, all 100% MCC, but differing in particles size. The CELPHERE® MCC bead grade represents the target particle size distribution for a given product (e.g., CP-102 bead size between 100-200 microns, CP-507 bead size between 500-700 microns, etc.). In all cases, the CELPHERE® MCC bead substrate was coated to a target amantadine weight gain of 50% w/w. Potency of all drug-layered CELPHERE® MCC beads was confirmed via gas chromatography. Each of the drug-loaded bead lots was then coated with the same coating formulation (ETHOCEL®10:DEP=4:1; (95% absolute ethanol/5% water) to the same target weight gain (20% w/w). The end result was five different lots of coated beads having differing particle size distributions but otherwise identical compositions.

Dissolution results for each of the coated bead lots are shown in FIG. 4, along with the corresponding formulation table. The finer grades of coated beads showed much faster release of amantadine when compared to coarser grades. The data suggest that bead particle size distribution is likely an important factor in providing adequate release retardation for a highly soluble active such as amantadine hydrochloride.

| Lot # | 224.072 | 224.076 | 224.080 | 224.099 | 224.103 |
|---|---|---|---|---|---|
| Substrate | 83.33 | 83.33 | 83.33 | 83.33 | 83.33 |
| Amantadine HCl | 41.67 | 41.67 | 41.67 | 41.67 | 41.67 |
| PVP K30 | 4.17 | 4.17 | 4.17 | 4.17 | 4.17 |
| Celphere CP-102 | 37.5 | | | | |
| Celphere CP-203 | | 37.5 | | | |
| Celphere CP-305 | | | 37.5 | | |
| Celphere CP-507 | | | | 37.5 | |
| Celphere CP-708 | | | | | 37.5 |
| Coating | 16.67 | 16.67 | 16.67 | 16.67 | 16.67 |
| Ethocel 10 | 13.34 | 13.34 | 13.34 | 13.34 | 13.34 |
| DEP | 3.33 | 3.33 | 3.33 | 3.33 | 3.33 |

Closer inspection of the dissolution profile from coated beads comprising CP-708 bead cores (bead core size between 700-800 microns) reveals that a release profile yielding 80% released at 24 hours was achieved. Also, the dissolution profile for these coated beads is nearly linear, indicating zero-order release kinetics. In general, it appears that the release profiles appear to flatten-out (approach zero-order) with increasing particle size.

### Example 4

### Thickness of Coating Layer

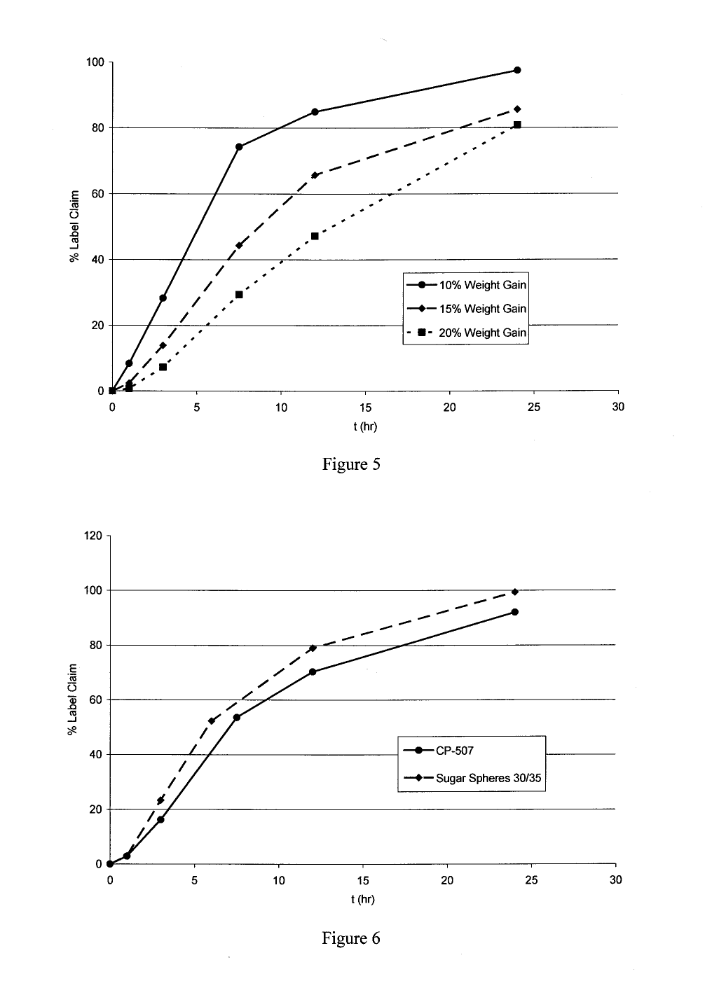

FIG. 5 shows additional release profiles for drug-loaded CELPHERE®MCC CP-708 bead cores (Lot 224.103 composition in table above) coated with an ethylcellulose-based coating (ETHOCEL®10) to varying weight gains. As FIG. 5 demonstrates, the coating layer weight gain (which relates to coating thickness) has a significant impact on amantadine release, with increasing weight gains showing slower release.

### Example 5

### Bead Core Type

To investigate the impact of substrate type on amantadine release, two sets of coated beads of nearly identical particle size distribution were compared. Sieve analysis demonstrated that the sugar spheres 30/35 and CELPHERE® MCC bead CP-507 have a nearly identical particle size distribution, both prior to, and post, drug layering. FIG. 6 shows dissolution profiles of identical ethylcellulose-based coating layers (ETHOCEL®10) on sugar spheres (Lot 224.060) and CELPHERE® MCC bead CP-507 (Lot 224.099). The coated bead lots differ in substrate type only.

The data suggest that the chemical make-up of the inert substrate does not play a critical role in controlling amantadine release kinetics from these coated beads. Bead core size appears to be more important for achieving slower release kinetics for the ethylcellulose-based coating system, suggesting that osmotic differences between the two substrates are negligible or are masked by the high osmotic pressure resulting from large concentration of amantadine (50% w/w) beneath the ethylcellulose-based coating layer.

### Example 6

### Stability of Multiparticulate Formulations

Three different formulations were manufactured in order to evaluate stability under controlled environmental conditions. It was desirable to demonstrate 6-month stability under accelerated conditions (40° C./75% RH) as this threshold is often used to justify product shelf-lives of up to two years at ambient conditions.

Multiple batches of drug-layered beads were manufactured to support stability studies. CELPHERE® microcrystalline cellulose spheres (Asahi Kasei Chemical Corporation) served as the inert substrate for drug layering. The two grades of spheres had a rated particle size range of 500-710 or 710-850 microns. The drug-layering solution was made with a drug:polymer ratio of 10:1 and a solution viscosity suitable for spraying. Polyvinylpyrrolidone (PVP, KOLLIDON® 30, BASF) was used as a drug binder. The drug-layering solution had the following composition (% weight/weight or % w/w):

| | |
|---|---|
| 32.0% w/w | Amantadine HCl |
| 3.2% w/w | PVP (KOLLIDON ® 30) |
| 64.8% w/w | Water |

Drug-loaded beads were prepared by fluidized bed coating. In all cases, the target drug load was 50% w/w and was confirmed by gas chromatography.

The drug-layered beads were then coated with a sustained-release polymer system. Ethylcellulose NF (ETHOCEL® 10, DOW Chemical Company) was chosen, as this polymer was demonstrated in previous studies to have desirable release properties. Diethyl phthalate was used as a plasticizer in the coating solution. The coating solvent was 95% ethanol, 5% water. Batches were coated to a final weight gain of either 10% or 20% using a fluidized bed in a Wurster column configuration. A formulation summary can be found below:

Composition of Coated Beads for Stability Evaluation

| | Lot # | | |
|---|---|---|---|
| | Formulation 1 | Formulation 2 | Formulation 3 |
| Substrate | 90.91 | 83.33 | 83.33 |
| Amantadine HCl | 45.45 | 41.67 | 41.67 |
| PVP K30 | 4.55 | 4.17 | 4.17 |
| Celphere CP-507 | — | — | 37.50 |
| Celphere CP-708 | 40.91 | 37.50 | — |
| Coating | 9.09 | 16.67 | 16.67 |
| Ethocel 10 | 7.27 | 13.33 | 13.33 |
| DEP | 1.82 | 3.33 | 3.33 |

Note:
all composition table values are in % weight/weight

After coating, each batch was sized using an ATM sonic sifter and then sieved through USA Standard test sieves. The coated beads were passed through a #16 size sieve to separate any agglomerates. Useable beads were retained on a size #50 size sieve.

For a given lot number, coated beads were filled into size “00CS” hard gelatin capsules (Capsugel, white opaque). For initial testing, capsules were filled gravimetrically (by hand) to achieve a label claim of 200 mg and evaluated. Subsequently, stability samples were manufactured using a Profill apparatus. Capsules were filled to volume and the weight of each capsule was checked. A target capsule weight range was determined and samples greater than or less than 2% of target were discarded. Label claim values were determined by fill weight bead drug load.

Final Capsule Formulations Manufactured Using Profill Apparatus

| | | Bead Drug | Label Claim |
|---|---|---|---|
| | Fill Weight | Load | (mg/cap) |
| Formulation 1 | 690 | 0.4545 | 315 |
| Formulation 2 | 659 | 0.4167 | 275 |
| Formulation 3 | 645 | 0.4167 | 270 |

Each lot of capsules was manually filled into 60 cc, white, opaque, HDPE bottles. No desiccant was added. Bottles were sealed with foil induction seals and capped with 30 mm plastic caps. This is a typical packaging configuration for solid, oral, dosage formulations.

Samples were stored in the sealed HDPE bottles for 6 months at 40° C./75% RH. Stability samples were evaluated at initial, 1-, 2-, 3- and 6-month timepoints. A second set of samples was stored at 25° C./60% RH. This was a back-up storage condition that would only be tested if significant changes were seen at the accelerated condition.

### Analytical Methods

Appearance and dissolution testing was done at all timepoints. Assay and water testing was completed at selected timepoints.

Appearance: Samples were evaluated visually to ensure the integrity of the white, opaque, size 00 capsule shells. Capsules shells were opened to ensure the contents continued to appear as white to off-white polymer coated beads. The beads were verified to be free flowing.

Visual assessment of samples at each time point was acceptable. No color changes were noted and in all cases the beads remained free-flowing.

Assay: Samples were assayed to determine the extent of degradation of active ingredient over time.

Diluent was prepared by mixing equal amounts of methanol and water. Solvents were high purity (HPLC grade). Internal standard solution (“ISTD solution”) was prepared by dissolving approximately 240 mg naphthalene in exactly 1 liter of hexane (HPLC grade).

One sample capsule was emptied into a 100-mL volumetric flask. Approximately 70 mL of diluent was added. Samples were shaken on a lateral shaker for four hours, then filled to the mark with diluent. Into a separate 100-mL volumetric flask, 5.0 mL of the sample solution was accurately pipetted. 2.0 mL of 5N NaOH was added, then mixed by hand. 50.0 mL of the ISTD solution was accurately pipetted into the same 100-mL volumetric flask, and the flask was shaken using a lateral shaker for one hour. The hexane layer was assayed. GC vials were filled directly with no filtration.

Samples were assayed by external chromatographic standards. To prepare standards, approximately 250 mg amantadine was added into a 100-mL volumetric flask. Approximately 70-mL of diluent was added and shaken for one hour on a lateral shaker, and the flask was filled to the mark with diluent.

For the final standard preparation, 5.0 mL of standard stock was added into a 100-mL vol. flask. 2.0 mL of 5N NaOH was added and mixed by hand. 50 mL ISTD solution was added and shaken for 1 hour on lateral shaker. The hexane layer was assayed. GC vials were filled directly with no additional filtration.

The GC parameters used for the dissolution procedure were also used for the assay testing. The naphthalene peak was well resolved from the amantadine peak in the chromatograms. External standards bracketed samples.

Results of the assay testing are given in the table below. Assay of the 3-month and 6-month stability samples demonstrated acceptable values that were within the target range of 90%-110% of label claim. No evidence of degradation was seen in the chromatograms.

Results of Assay and Degradation Product Testing For Aged Samples

| | 3 month | 6 month |
|---|---|---|
| Formulation 1 | 97.9 | 96.8 |
| Formulation 2 | 95.8 | 97.3 |
| Formulation 3 | 98.0 | 100.1 |

Note 1:
no evidence of degradation observed in chromatograms
Note 2:
typical specification would be 90%-110% of label claim

Karl Fischer (water content): Samples were tested to determine changes in water content over time. The contents of 3-4 capsules were ground with a mortar and pestle. An aliquot of 200-400 mg was titrated using an automated Karl Fischer titrator (Metrohm model 784) with standardized titrant.

Results are given in the table below. Water content testing of selected stability samples by Karl Fischer titration yielded acceptable results and showed the formulations to be non-hygroscopic.

Results of Karl Fischer Testing For Aged Samples

| | Initial | 3 month | 6 month |
|---|---|---|---|
| Formulation 1 | 0.4 | 1.6 | 0.7 |
| Formulation 2 | 0.5 | 1.5 | 0.6 |
| Formulation 3 | 0.4 | 1.4 | 0.5 |

Dissolution: Formulations for the stability study were selected for dissolution testing, to determine whether the aged samples would meet targets for release at a specified time point. The targets ranged from NMT (not more than) 40% to NMT 80% of the label claim released at the 6-hour time point. The previously described procedure for dissolution of amantadine coated beads was used. Single capsules were placed in each basket without a sinker. For each stability time point, six capsules were evaluated. GC parameters have been described. Dissolution testing employed bracketing chromatographic standards and the memantine internal standard.

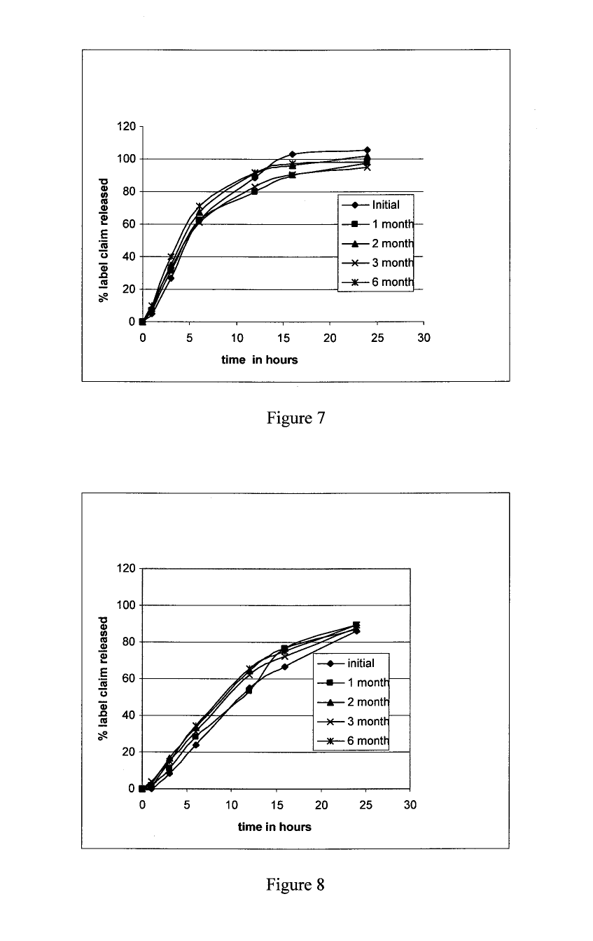

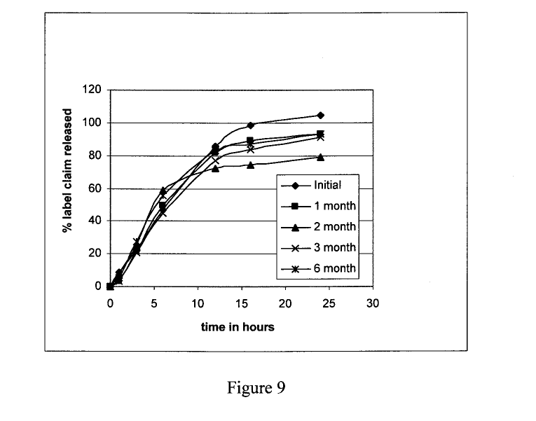

6-hour data from the initial testing through 6-month stability testing is summarized in the table below. The results demonstrate the acceptability of the formulations in meeting the target criteria at the 6-hour time point. No clear trends in the 6-hour data were seen over time. The scatter observed in the dissolution data is believed to be related to the precision and ruggedness of the analytical method. Additionally, a review of the overall dissolution profiles (FIGS. 7-9) shows no gross shifts to the in vitro profiles, thus demonstrating the integrity of the coated beads under the accelerated storage conditions.

Summary of 6-Hour Timepoint Dissolution Data For Aged Samples

| | | % LC range | Mean of data | |
|---|---|---|---|---|
| | Target at 6 | at 6 hours | at 6 hours | Standard |
| Formulation | hours | observed | (n = 5) | Deviation |
| 1 | NMT | (61.3-71.4) | 65.0% LC | 4.3 |
| | 80%@6 hr | | | |
| 2 | NMT | (23.8-34.6) | 30.2% LC | 4.3 |
| | 40%@6 hr | | | |
| 3 | NMT | (45.1-58.7) | 51.1% LC | 5.8 |
| | 60%@6 hr | | | |

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A dosage unit for providing extended release of a highly soluble pharmacologically active compound, the dosage unit comprising a plurality of coated beads, wherein each coated bead comprises:
a bead core having a diameter of about 500 microns or larger,
a drug layer applied to the bead core, the drug layer comprising the highly soluble pharmacologically active compound; and
a coating layer substantially encapsulating the bead core and drug layer and adapted to retard release of the pharmacologically active compound when the coated beads are exposed to an aqueous or acidic environment, wherein the coating layer comprises an ethylcellulose polymer.

2. The dosage unit of claim 1, wherein the pharmacologically active compound is a salt of amantadine or rimantadine.

3. The dosage unit of claim 1, wherein the pharmacologically active compound is amantadine hydrochloride.

4. The dosage unit of claim 3, wherein the dosage unit comprises from about 50 mg to about 400 mg of amantadine hydrochloride.

5. The dosage unit of claim 1, wherein about 10 wt.-% to about 50 wt.-% of the coated bead is the pharmacologically active compound.

6. The dosage unit of claim 1, wherein the bead core is an inert sugar sphere or a microcrystalline cellulose seed core.

7. The dosage unit of claim 1, wherein the dosage unit comprises bead cores having a diameter from about 710 to about 850 microns.

8. The dosage unit of claim 1, wherein about 50 wt.-% to about 95 wt.-% of the drug layer is the pharmacologically active compound.

9. The dosage unit of claim 1, wherein the drug layer further comprises a binder.

10. The dosage unit of claim 1, wherein from about 10% wt.-% to about 30 wt.-% of the coated bead is the coating layer.

11. The dosage unit of claim 1, wherein the coating layer further comprises a plasticizer.

12. The dosage unit of claim 1, wherein the coating layer retards release of the pharmacologically active compound when the coated beads are exposed to an aqueous or acidic environment such that not more than 50 wt.-% of the total pharmacologically active compound of the dosage unit is released into the environment within 4 hours.

13. The dosage unit of claim 1, wherein at least 80 wt.-% of the total pharmacologically active compound of the dosage unit is released into the environment within 24 hours.

14. The dosage unit of claim 1, wherein at least 80 wt.-% of the total pharmacologically active compound of the dosage unit is released into the environment within 12 hours.

15. The dosage unit of claim 1, wherein at least 80 wt.-% of the total pharmacologically active compound of the dosage unit is released into the environment within 8 hours.

16. The dosage unit of claim 1 in the form of a capsule, a tablet, or a sachet.

17. The dosage unit of claim 1, wherein the pharmacologically active compound is a salt of amantadine, the bead core is an inert sugar sphere or a microcrystalline cellulose seed core, the drug layer further comprises polyvinylpyrrolidone, and the coating layer further comprises diethyl phthalate.

18. A method for providing a dosage unit that provides extended release of a highly soluble pharmacologically active compound, the dosage unit comprising a plurality of coated beads, the method comprising the steps of:
applying a drug layer to a plurality of bead cores, each having a diameter of about 500 microns or larger, wherein the drug layer comprises the highly soluble pharmacologically active compound; and
encapsulating the drug layer substantially within a coating layer to provide the coated beads, the coating layer adapted to retard release of the pharmacologically active compound when the coated beads are exposed to an aqueous or acidic environment, wherein the coating layer comprises an ethylcellulose polymer.

19. A method of treating a patient having Parkinson's Disease, comprising administering to the patient the dosage unit of claim 1, wherein the pharmacologically active compound is a salt of amantadine.

20. A method of treating a patient having a viral infection, comprising administering to the patient the dosage unit of claim 2.
